(19) United States
(12) Patent Application Publication
(10) Pub. No.: US 2021/0084860 A1
(43) Pub. Date: Mar. 25, 2021

(54) "Prunus Rootstock Named 'Dhprs2'"

(71) Applicant: Michael Neumüller, Hallbergmoos (DE)

(72) Inventor: Michael Neumüller, Hallbergmoos (DE)

(21) Appl. No.: 17/024,945

(22) Filed: Sep. 18, 2020

Related U.S. Application Data
(60) Provisional application No. 62/973,196, filed on Sep. 20, 2019.

(51) Int. Cl. A01H 6/74; A01H 5/08

(52) U.S. Cl. CPC A01H 6/7427; A01H 5/08

(57) ABSTRACT

A new and distinct prunus variety used as a rootstock that exhibits desirable disease resistance and induces a high yield of large fruits for grafted varieties.

### CROSS REFERENCE TO RELATED APPLICATION

This application claims the benefit of U.S. Provisional Patent Application Ser. No. 62/973,196, filed on Sep. 20, 2019, and entitled “Prunus Rootstock Named ‘Dhprs2’”, the entire disclosure of which is incorporated herein by reference.

### BOTANICAL CLASSIFICATION

Prunus domestica x Prunus spinosa

### VARIETAL DENOMINATION

‘Dhprs2’

### BACKGROUND OF THE INVENTION

The present invention comprises a new and distinct cultivar of (Prunus domestica x Prunus spinosa) used as a rootstock and known by the varietal name ‘Dhprs2’. The new variety is the result of a planned breeding program conducted in Freising, Germany. The new variety is the result of a cross between Prunus domestica variety named ‘Jojo’ (female parent, unpatented) and an unnamed Prunus spinosa variety (male parent, unpatented). The new variety was first asexually reproduced via in vitro propagation in Freising, Germany.

The new variety has not been evaluated under all possible environmental conditions. The phenotype may vary with variations in environment without a change in the genotype of the plant. The new variety has been trial and field tested and has been found to retain its distinctive characteristics and remain true to type through successive asexual propagations.

### DESCRIPTION OF THE DRAWINGS

The accompanying photographic drawings illustrate the new variety at approximately 4 years of age, with the color being as nearly true as possible with color illustrations of this type.

FIG. 1 illustrates numerous plants of ‘Dhprs2’;

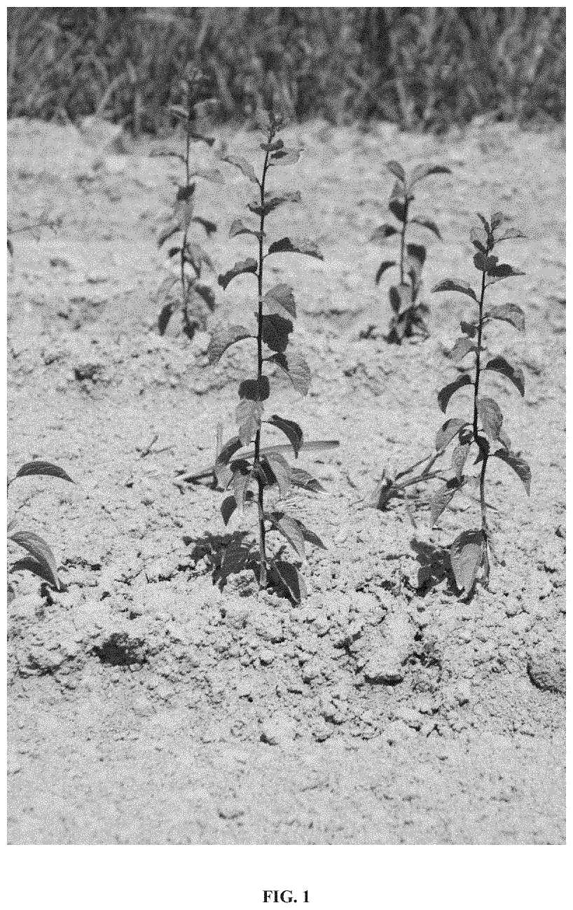

FIG. 2 illustrates the flowers of ‘Dhprs2’; and

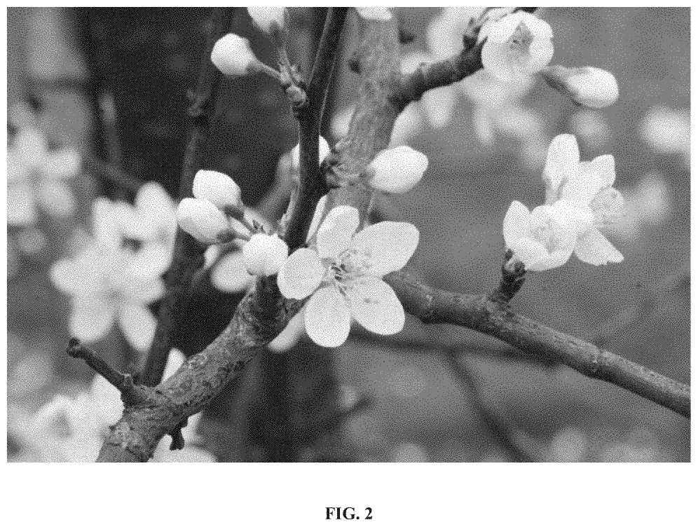

FIG. 3 illustrates the fruit of ‘Dhprs2’.

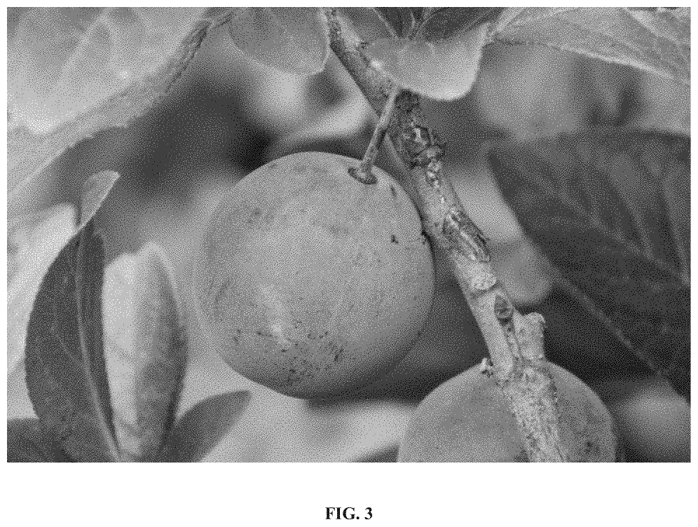

### DETAILED BOTANICAL DESCRIPTION

The following detailed description sets forth the characteristics of the new cultivar at approximately 6 years of age. The data which defines these characteristics were collected as a result of asexual reproductions carried out in Freising, Germany. ‘Dhprs2’ is compatible with at least varieties of Prunus domestica and Prunus salicina and induces a high yield of large fruits for grafted varieties. ‘Dhprs2’ is fully resistant to the Plum pox virus (PPV/Sharka) by means of hypersensitivity resistance. Further, the new variety exhibits good resistance to plum decline caused by Pseudomonas bacteria. ‘Dhprs2’ exhibits medium vigor (similar to prunus rootstock variety named ‘Weiwa’—unpatented) with only a few suckers present. Its leaf blades are narrow, serrate, elliptical in shape, and of medium length. Its flower petals are pure white in color. ‘Dhprs2’ can be useful as a rootstock for grafting of PPV resistant varieties, as well as with varieties that are PPV sensitive. Color references are primarily to the R.H.S. Colour Chart of The Royal Horticultural Society of London and were identified under natural light.

### Rootstock

Size: Medium.
Vigor: Moderate.
Pathogen resistance: Resistant to the Plum pox virus (PPV/Sharka) and good resistance to Pseudomonas bacteria.
Rootstock performance:
   Root sprouts (suckering): Few.
   Anchorage: Good.
   Compatibility: Very good with Prunus domestica; also compatible with Prunus salicina.
   Vigor: Moderate (70% with Prunus cerasifera).
Branches:
   Diameter: 5-18 mm.
   Color: RHS N200C.
   Lenticels:
      Length: 0.9-1.2 mm
      Width: 0.1-0.2 mm.
      Color: RHS N199C.
Leaves:
   Length: 64-70 mm.
   Width: 42-49 mm.
   Form: Elliptic.
   Base: Obtuse-truncate.
   Apex: Apiculate.
   Margin: Crenate-serrate.
   Mature leaf color:
      Upper surface: RHS 137A.
      Lower surface: RHS 137C.
   Petiole:
      Length: 11-14 mm.
      Diameter: 1.5-1.8 mm.
      Color: RHS N77A.
   Veins:
      Venation type: Reticulate.
      Color: RHS 141D.
Tree winter hardiness: High.
Bud winter hardiness: High.
Drought tolerance: Good.

I claim:

1. A new and distinct variety of prunus rootstock plant, as illustrated and described herein.
